(19) United States
(12) Patent Application Publication
(10) Pub. No.: US 2025/0107841 A1
(43) Pub. Date: Apr. 3, 2025

(54) MULTI-LAYER ANTI-STICK COATING FOR SURGICAL TOOLS

(71) Applicant: Cilag GmbH International, Zug (CH)

(72) Inventors: Luling Wang, Mason, OH (US); Gregory A. Trees, Cincinnati, OH (US); Candice Otrembiak, Loveland, OH (US); John Michael Sarley, Mason, OH (US)

(21) Appl. No.: 18/896,233

(22) Filed: Sep. 25, 2024

Related U.S. Application Data
(60) Provisional application No. 63/586,231, filed on Sep. 28, 2023.

(51) Int. Cl. A61B 18/14; A61B 18/00

(52) U.S. Cl. CPC A61B 18/1445; A61B 2018/00077; A61B 2018/0013; A61B 2018/00136

(57) ABSTRACT

Various exemplary devices, systems, and methods for multi-layer anti-stick coatings for surgical tools are provided. In general, a method of manufacturing a surgical tool includes forming, using plasma enhanced chemical vapor deposition with hexamethyldisiloxane as a precursor material, a first coating on a conductive tissue treating surface of the end effector and a second jaw component of the end effector, the first coating comprising a first material, wherein the first material is a first silicone material, and applying a second coating on top of the first coating on the first jaw component and the second jaw component, the second coating comprising a second material, wherein the coatings are effective to prevent tissue sticking to the jaws during an electrosurgical sealing procedure.

### RELATED APPLICATION

This application claims priority under 35 U.S.C. § 119(e) to U.S. Provisional Application No. 63/586,231 filed Sep. 28, 2023, the entire contents of which are hereby expressly incorporated by reference herein.

### FIELD

The present disclosure generally relates to multi-layer anti-stick coating for surgical tools.

### BACKGROUND

Various types of surgical devices are used for grasping, cutting, and sealing tissue. In general, these devices have jaws configured to grasp tissue and a cutting mechanism configured to be advanced through the tissue to sever it. Some of these devices can apply energy to the tissue disposed between the jaws to promote hemostasis. In instances where the tissue includes a vessel, these devices can also apply energy to the tissue disposed between the jaws to seal the vessel.

A common issue when using one of these surgical devices is that the jaws, or other components of the surgical device, sticks to the tissue that the device is grasping, cutting, sealing, or otherwise treating. Tissue stuck to a surgical device may be pulled out of place and/or damaged when removing the surgical device from the tissue. In some instances, this damage may compromise the treatment performed by the surgical device.

Attempts have been made in the art to provide surgical devices that include various non-stick surface coatings. For example, U.S. Pat. Nos. 10,709,497 and 10,973,569, both assigned to Covidien LP, discuss various polydimethylsiloxane non-stick coatings. However, these patents teach that if the applied non-stick coating is too thin (e.g., less than about 20 nm), the non-stick coating may not provide adequate tissue sticking reduction, and if the non-stick coating is above a particular thickness (e.g., greater than about 200 nm) this may create a uniform dielectric barrier or surface impedance on the sealing plates.

Accordingly, there remains a need for improved devices, systems, and methods for reducing sticking between surgical tools and tissue these tools contact.

### SUMMARY

In general, devices, systems, and methods for multi-layer anti-stick coating for surgical tools are provided.

In one aspect, a method of manufacturing a surgical tool is provided. In one embodiment, the method includes forming, using plasma enhanced chemical vapor deposition (PECVD) with a precursor material, a first coating on a conductive tissue treating surface of a component of the end effector, the first coating comprising a first material, wherein the first material is a first silicone material, and applying a second coating on top of the first coating on the conductive tissue treating surface, the second coating comprising a second material, wherein the coatings are effective to prevent tissue sticking to the conductive tissue treating surface during an electrosurgical sealing procedure.

The method can vary in any number of ways. For example, the precursor used for plasma enhanced chemical vapor deposition is hexamethyldisiloxane.

For another example, the first silicone material is a polydimethylsiloxane-like material.

For still another example, the first silicone material comprises polydimethylsiloxane.

For another example, the second material comprises a phospholipid material.

For yet another example, the second material comprises a second silicone material different from the first silicone material. In some instances, the second silicone material comprises an amino-functional silicone.

For still another example, applying the second coating comprises wiping the second material onto the conductive tissue treating surface.

For another example, applying the second coating comprises spraying the second material onto the conductive tissue treating surface.

For yet another example, applying the second coating comprises brushing the second material onto the conductive tissue treating surface.

For still another example, applying the second coating comprises dipping the conductive tissue treating surface into the second material.

For another example, the component is a first jaw component of the end effector. In some instances the method also includes after forming the first coating and prior to applying the second coating, assembling the end effector using the first jaw component and a second jaw component. In some instances the method also includes after applying the second coating, assembling the end effector using the first jaw component and a second jaw component.

For yet another example, the first coating has a thickness of approximately 7 to 17 nm or 220 to 300 nm. In some instances, the first coating has a thickness of approximately 7 to 17 nm. In some instances, the first coating has a thickness of approximately 220 to 300 nm.

For another example, the second coating has a thickness of 300 nm to 5 μm.

For still another example, the second coating has a thickness of 0.1-1 μm over approximately 50-95% of the sealing surface area to which the second coating has been applied, and a thickness of greater than 7 μm over less than 5% of the sealing surface area to which the second coating has been applied.

According to another aspect, an end effector of an electrosurgical device is provided. In one embodiment, the end effector includes a first jaw component having a conductive first tissue treating surface, a second jaw component operatively coupled to the first jaw component, the second jaw component having a conductive second tissue treating surface, a first coating on the conductive first tissue treating surface and the conductive second tissue treating surface, the first coating comprising a first material that is a first silicone material, and a second coating layered on top of the first coating, the second coating comprising a second material.

The end effector can vary in a number of ways. For example, the first silicone material can be derived from hexamethyldisiloxane.

In another example, the first silicone material includes polydimethylsiloxane.

In yet another example, the second material includes a phospholipid material.

In still another example, the second material includes a second silicone material different from the first silicone material.

In yet another example, the first coating has a thickness of approximately 7 to 17 nm or 220-300 nm. In some instances, the first coating has a thickness of approximately 7 to 17 nm. In some instances, the first coating has a thickness of approximately 220 to 300 nm.

In another example, the second coating has a thickness of 300 nm to 5 μm.

In still another example, the second coating has a thickness of 0.1-1 μm (or 0.2-1 μm) over approximately 50-95% of the sealing surface area to which the second coating has been applied, and a thickness of greater than 7 μm over less than 5% of the sealing surface area to which the second coating has been applied.

In another example, the first coating is a plasma enhanced chemical vapor deposited material.

In yet another example, the first jaw component and the second jaw component have a curved shape.

In another example, the first jaw component and the second jaw component have a straight shape.

In another example, the first coating covers a larger surface area on the first jaw component and the second jaw component than the second coating.

### BRIEF DESCRIPTION OF THE DRAWINGS

This disclosure will be more fully understood from the following detailed description taken in conjunction with the accompanying drawings, in which:

FIG. 1 is a schematic cross-sectional view of a portion of a surgical tool with a multi-layer anti-stick coating;

FIG. 2 is a perspective view of an exemplary surgical tool coated with the multi-layer anti-stick coating of FIG. 1;

FIG. 3 is a perspective view of the end effector of the surgical tool of FIG. 2;

FIG. 4 is a schematic of a process for forming the multi-layer coating of FIG. 1 on the surgical tool of FIG. 2;

FIG. 5A is a schematic of a vapor deposition system for creating a first layer of the multi-layer coating of FIG. 1;

FIG. 5B is a flow chart showing a process for creating a first layer of the multi-layer coating of FIG. 1;

FIG. 5C is a Fourier Transform Infrared spectroscopy output showing the spectrum of a PDMS-like coating used as a first layer of the multi-layer coating of FIG. 1;

FIG. 6A is a perspective view of a dipping process for applying a second layer of the multi-layer anti-stick coating of FIG. 1; and

FIG. 6B is a perspective view of a wiping process for applying a second layer of the multi-layer anti-stick coating of FIG. 1.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices, systems, and methods specifically described herein and illustrated in the accompanying drawings are nonlimiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Sizes and shapes of the systems and devices, and the components thereof, can depend at least on the anatomy of the subject in which the systems and devices will be used, the size and shape of components with which the systems and devices will be used, and the methods and procedures in which the systems and devices will be used.

Various exemplary devices, systems, and methods for manufacturing multi-layer anti-stick coatings for surgical tools are provided. In general, during a surgical procedure, a surgical tool contacts tissue to manipulate and/or repair and/or coagulate the tissue. During or after manipulation and/or repair and/or coagulation of tissue, the surgical tool is separated from the tissue. The devices and methods described herein provide the ability to separate the surgical tool from the tissue without damaging the tissue and/or moving the tissue out of position due to sticking. The multi-layer anti-stick coating has a first layer including a silicone or a silicone-like material and a second layer including a phospholipid or silicone material. The first layer is applied to an area of the surgical tool, or its components prior to assembly, using a plasma enhanced chemical vapor deposition process. The second layer is applied via a dipping, wiping, brushing, and/or spraying process and covers at least a portion of the area of the surgical tool covered by the first layer or a portion of areas covered by the first layer.

The systems, devices, and methods described herein have applicability in a variety of surgical procedures that require energy to be delivered to the tissue including vessel repair and/or coagulation, soft tissue repair and/or coagulation, and/or tissue manipulation, such as laparoscopic procedures using advanced bipolar RF energy devices, ultrasonic laparoscopic surgical devices, or open surgery using monopolar devices.

During surgical procedures, especially electrosurgical procedures, tissue sticking to surgical tools used to perform the surgery can cause interruption of the procedure and sometimes can cause damage to the tissue. Therefore, it is important that the surgical tools used during the procedure not stick to the tissue being treated. Anti-stick coatings applied to surgical tools, for example, on conductive tissue-treating surfaces of electrosurgical tools, such as the multi-layer anti-stick coatings discussed herein, can prevent tissue from sticking to surgical tools. It is also important that the anti-stick coating not impair the functionality of the surgical tools, for example, the ability to deliver energy to tissue to seal/coagulate the tissue. The multi-layer anti-stick coatings discussed herein allow for electrosurgical energy to pass therethrough to enable effective electrosurgical treatment.

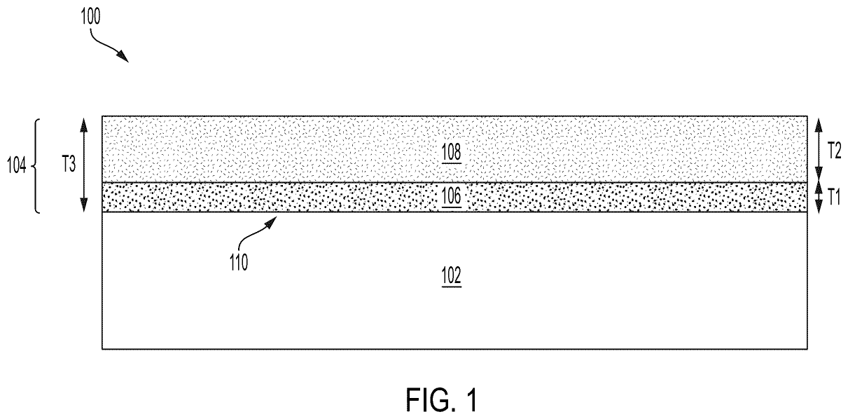

FIG. 1 is a cross-sectional view of a portion 100 of a surgical tool 102 with a multi-layer anti-stick coating 104 that is formed on a conductive, tissue-contacting surface of the surgical tool 102. The multi-layer anti-stick coating 104 includes a first coating 106 and a second coating 108, each of these coatings forming a layer of the multi-layer anti-stick coating 104. In some embodiments, the tissue treating surface is a conductive tissue treating surface configured to transmit electrical energy to tissue during an electrosurgical procedure. In some embodiments, the second coating 108 covers a portion of the area covered by the first coating 106. In some embodiments, the second coating 108 covers all of the area covered by the first coating 106.

In the embodiment of FIG. 1, the first coating 106 is formed of a silicone or silicone-like material. In some embodiments, the silicone material is a polydimethylsiloxane-like material that is derived from hexamethyldisiloxane. In a preferred embodiment, the first coating 106 has a thickness T1 that is between approximately 7 and 17 nm, preferably approximately 15 nm. In some embodiments, the first coating has a thickness of 7, 8, 9, 10, 11, 12, 13, 14, 15, 16, or 17 nm. In another preferred embodiment, the first coating 106 has a thickness T1 that is between approximately 220 and 300 nm. In some embodiments, the first coating 106 has a thickness T1 that is approximately 220, 230, 240, 250, 260, 270, 280, 290, or 300 nm. In some embodiments, the first coating 106 has a thickness T1 of between approximately 7 and 20 nm or between approximately 200 and 300 nm.

In the embodiment of FIG. 1, the second coating 108 is formed of a material different from the material forming the first coating 106. In some embodiments, the second coating 108 is formed from a phospholipid material. In some embodiments, a commercially-available phospholipid material may be used as a coating layer and may be obtained, for example, from Electrolube (Key Surgical), Sigma-Aldrich, Lipoid, Cargill Lecithin, NOF America, etc. In some embodiments, the second coating 108 is formed from a silicone material different from the silicone material of the first coating 106. The silicone material for the second coating 108 may be, for example, an amino-functional silicone applied by dip, spray, brush, or wipe. The silicone coating may cure based on moisture and/or temperature, may have a catalyst to help with curing and reaction, may be a silane functionalized silicone, or a photocurable silicone or any combination thereof. Some examples are included in book chapter “Medical Applications of Silicones” (Jim Curtis, Andre Colas, Chapter II.5.18—Medical Applications of Silicones, Editor(s): Buddy D. Ratner, Allan S. Hoffman, Frederick J. Schoen, Jack E. Lemons, Biomaterials Science (Third Edition), Academic Press, 2013, Pages 1106-1116, ISBN 9780123746269), U.S. Pat. Nos. 9,434,857, and 10,441,947, the contents of which are incorporated by reference herein in their entireties.

In some embodiments, when the second coating 108 is formed from a phospholipid material, the second coating 108 has a thickness T2 that is between approximately 300 nm and 5 μm, preferably 500 nm to 3 μm over at least a portion of the sealing surface of the surgical tool.

In some embodiments, at least 10, 20, 30, 40, 50, 60, 70, 80, 90, 95, 98, or 100 percent of the sealing surface of the surgical tool has a second coating with a thickness of approximately 300 nm to 5 μm, preferably 500 nm to 3 μm. In some embodiments, when the second coating 108 is formed from a silicone material applied via a dip, spray, brush, or wiping method, the second coating 108 has a thickness of 0.1-1 μm (or, in some embodiments, 0.2-1 μm) over approximately 50-95% of the sealing surface area to which the second coating has been applied, and a thickness of greater than 7 μm over less than 5% of the sealing surface area to which the second coating has been applied (e.g., less than 5%, 4%, 3%, 2%, or 1%). In certain embodiments, the second coating has a thickness of greater than 1 μm to 2 μm over approximately 3-10% or approximately 25-35% of the sealing surface area to which the second coating has been applied. In particular embodiments, the second coating 108 has a thickness distribution over the area to which the second coating has been applied that meets one of the examples shown in Table A below.

TABLE A
Second Coating Distribution

| | | |
|---|---|---|
| Second coating thickness | Example 1 | Example 2 |

| | | | |
|---|---|---|---|
| 0.1-1 | μm | 50-70% | 85-95% |
| >1-2 | μm | 20-35% | 3-10% |
| >2-7 | μm | 10-20% | 2-7% |
| >7 | μm | <5% | <5% |

Depending on the method used in applying the phospholipid coating, the thickness of the phospholipid coating may be variable over a surface on which the coating is applied. In some embodiments, at least 10, 20, 30, 40, 50, 60, 70, 80, 90, 95, 98, or 100 percent of the sealing surface of the surgical tool has a second coating with a thickness of 300 nm to 5 μm as previously discussed.

In some embodiments, the second coating 108 is formed from a silicone material using a dip, spray, brush, and/or wiping method. As will be understood by those skilled in the art, the coating thickness for dip coating can be controlled by solid percentage of coating materials, withdraw speed, soaking time, and drying process, and thickness for spray coating can be controlled by spray nozzle used, distance to the sample, solid percentage of coating materials and number of spray passes etc. One of skill in the art would understand that the coating thickness of second layer silicone can be controlled by process parameters and solution properties.

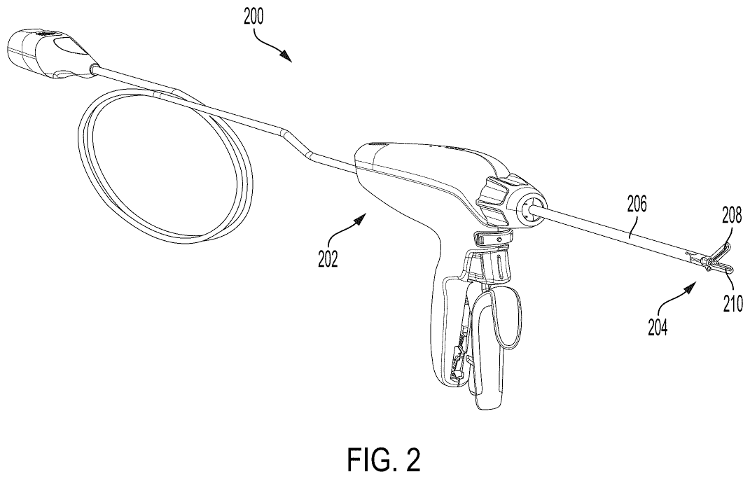

FIG. 2 is a perspective view of an exemplary surgical tool 200 coated with the multi-layer anti-stick coating 104 of FIG. 1. The surgical tool 200 is an electrosurgical vessel sealing tool that is configured to grab, seal, and cut tissue. The surgical tool 200 includes a proximal portion 202 which may be coupled to a handle for manual operation of the surgical tool or an adapter for use with a robotic surgical system for robotic operation of the surgical tool. The surgical tool 200 includes an end effector 204 at the distal end of a shaft 206. The end effector 204 includes a first jaw component 208 and a second jaw component 210. The jaws are configured to grab and hold tissue during operation of the surgical tool 200.

In some embodiments, instead of the vessel sealer surgical tool 200 shown in FIG. 2, the surgical tool may be a surgical stapler, surgical saw, a surgical drill, or another surgical implement or tool configured to interact with tissue. One of ordinary skill in the art would understand that the multi-layer anti-stick coatings described herein could be applied to any tissue-contacting tool, including any electrosurgical tool. One of ordinary skill in the art would understand that it may be advantageous to vary the thickness of the multi-layer anti-stick coating based on the surgical tool being coated. For example, surgical tools that do not deliver electrical energy via the coated surface (e.g., a conductive tissue treating surface) may be able to tolerate a thicker multi-layer anti-stick coating without compromising the performance of the surgical tool.

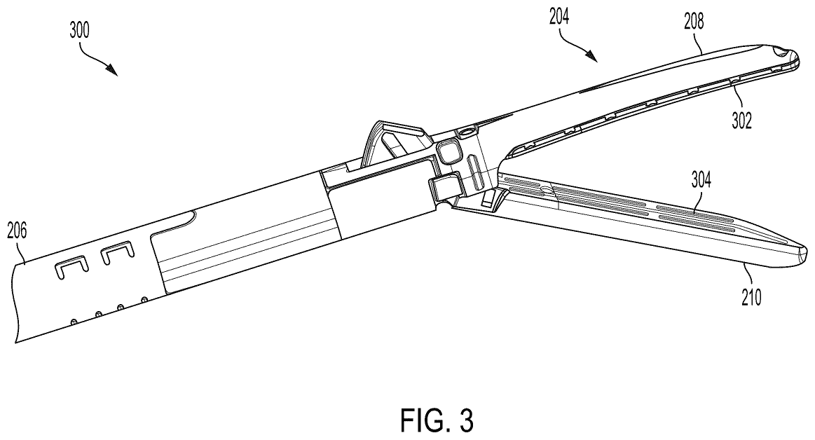

FIG. 3 is a perspective view of a distal portion 300 of the surgical tool 200 shown in FIG. 2. The surgical tool 200 is a tissue sealing tool configured to grab, seal, and cut tissue. The distal portion 300 includes a shaft 206 connected to an end effector 204. The end effector 204 of the surgical tool of FIG. 2. The end effector 204 includes a first jaw component 208 and a second jaw component 210 that form the jaws. The first jaw component 208 includes a first tissue treating surface 302 and the second jaw component 210 includes a second tissue treating surface 304. The first tissue treating surface 302 and the second tissue treating surface 304 are conductive surfaces configured to transmit electrical energy to tissue during an electrosurgical procedure. The first and second tissue treating surfaces 302, 304 are coated with the multi-layer anti-stick coating 104 shown in FIG. 1. In some embodiments, additional surfaces of the jaw components are coated with the first layer and/or the second layer of the anti-stick coating. In some embodiments, at least one of tissue treating surfaces 302, 304 is coated with the multi-layer anti-sticking coating. A conductive tissue treating surface may be formed from various conductive biocompatible materials, such as metal or metal composites.

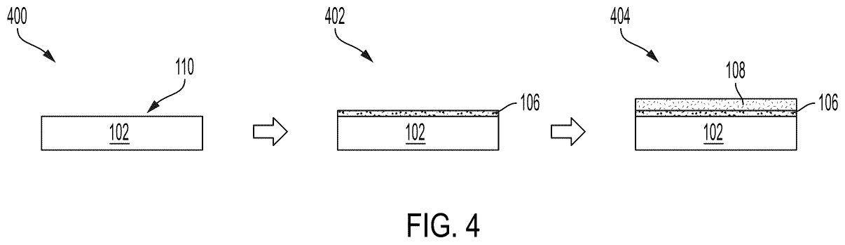

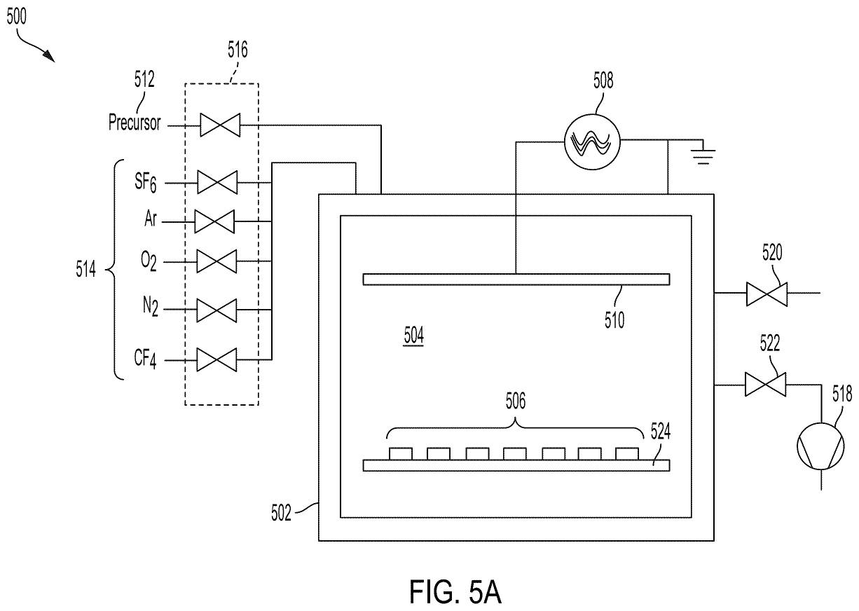

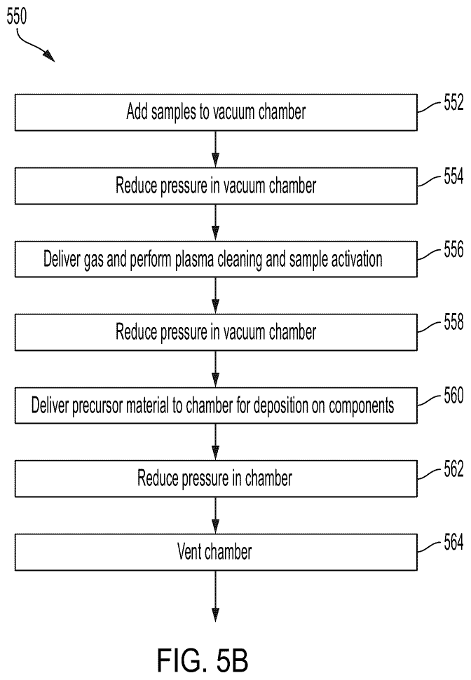

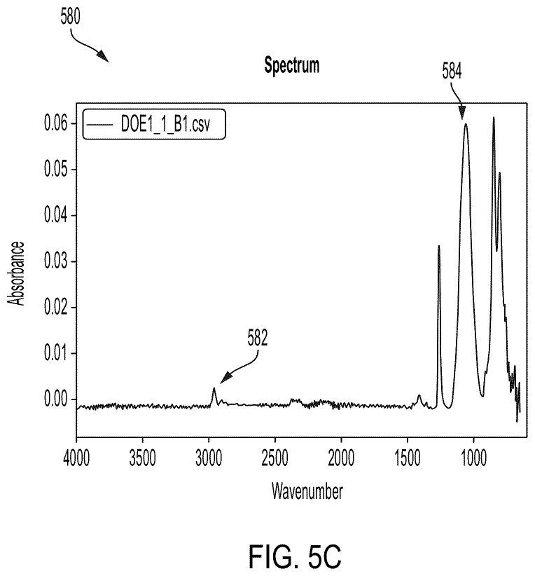

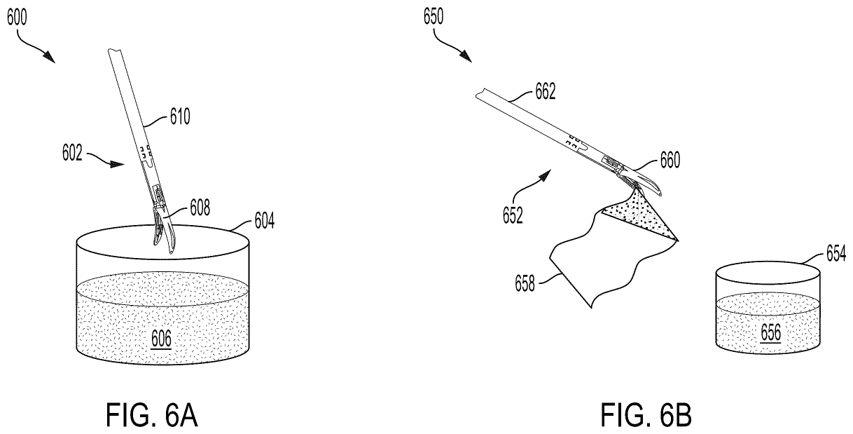

FIG. 4 is a schematic of a process of for forming the multi-layer coating of FIG. 1 on the surgical tool of FIG. 2. Generally, as shown at step 400, a tissue treating surface 110 of a surgical tool 102 is exposed. In a first process, shown at step 402, the tissue treating surface 110 is coated with a first coating 106. As described above with respect to FIG. 1, the first coating 106 is applied using a plasma enhanced chemical vapor deposition (PECVD) process and is a PDMS-like coating that has a thickness of between approximately 7 and 17 nm or 220 and 300 nm. In a preferred embodiment, the thickness of the first coating 106 is approximately 15 nm. An example apparatus and method for applying the first coating 106 is shown in FIGS. 5A-5B. In a second process, shown at step 404, a second coating 108 is applied on top of the first coating 106. The second coating 108 includes a phospholipid material or a silicone material and has a thickness of between 300 nm and 30 μm over at least a portion of the sealing surface. In some other embodiments, the second coating 108 has a thickness of approximately 300 nm to 5 μm, preferably 500 nm to 3 μm, over at least a portion of the sealing surface of the surgical tool. In some embodiments, at least 10, 20, 30, 40, 50, 60, 70, 80, 90, 95, 98, or 100 percent of the sealing surface of the thickness of the second coating 108 has a thickness of approximately 300 nm to 5 μm, preferably 500 nm to 3 μm. In some embodiments, the sealing surface of the thickness of the second coating 108 has a thickness of 0.1-1 μm (or, in some embodiments, 0.2-1 μm) over approximately 50-95% of the sealing surface area to which the second coating 108 has been applied, and a thickness of greater than 7 μm over less than 5% of the sealing surface area to which the second coating 108 has been applied (e.g., less than 5%, 4%, 3%, 2%, or 1%). In certain embodiments, the second coating has a thickness of greater than 1 μm to 2 μm over approximately 3-10% or approximately 25-35% of the sealing surface area to which the second coating108 has been applied. In particular embodiments, the second coating 108 has a thickness distribution over the area to which the second coating 108 has been applied that meets one of the embodiments in Table A. The second coating 108 may be applied via a variety of methods, including dipping, wiping, brushing, and/or spraying. Exemplary apparatuses and methods for applying the second coating 108 are shown in FIGS. 6A-6B.

FIG. 5A is a schematic of a system for creating a first layer of the multi-layer coating of FIG. 1. The plasma enhanced chemical vapor deposition system (PECVD) system 500 is used to produce polydimethylsiloxane (PDMS) like coatings on surgical tool components 506. In some embodiments, the PDMS-like coatings derived from hexamethlydisiloxane (HMDSO) are a silicone type material similar to PDMS. FIG. 5C shows Fourier Transform Infrared (FTIR) spectroscopy output 580 for the PDMS-like coating with peaks 582 and 584. One of skill in the art would understand that other chemical precursors may be used in the PECVD process to generate a similar PDMS-like structure for the anti-stick coatings described herein.

In some embodiments, the surgical tool components 506 are jaws of an end effector of a vessel sealer, for example, the end effector 204 of the surgical tool 200 shown in FIGS. 2-3. The PECVD system includes a vacuum chamber 502 that is configured to be filled with gas and plasma 504. In some embodiments, the plasma is an oxygen plasma for cleaning and activating the surface of the surgical tool components 506. In some embodiments, the gas includes sulfur hexafluoride, argon, oxygen, nitrogen, tetrafluoromethane, hydrogen, and/or other gases or gas combinations that can be used for plasma cleaning and activation. The pressure of the vacuum chamber 502 is controlled by a vacuum pump 518, vacuum valve 522, gas and/or precursor flow rates, and a vent valve 520. In some embodiments, the pressure of the vacuum chamber during the cleaning/activation and deposition steps is approximately between 25 mTorr and 750 mTorr. A person of skill in the art would understand that the vacuum pressure may be varied depending on the particular parameters of the PECVD system used. In the embodiment of FIG. 5A, the plasma 504 is an oxygen plasma. First, one or more surgical tool components 506 is placed in the vacuum chamber 502. In some embodiments, assembled surgical tools are placed in the vacuum chamber 502 prior to coating. In some embodiments, it is preferred to coat surgical tool components rather than assembled surgical tools due to a size and/or shape of the surgical tool. The vacuum chamber 502 is connected to a liquid precursor chemical 512, which may be, for example hexamethlydisiloxane (HMDSO). The vacuum chamber 502 is connected to process gases 514 as well as the precursor chemical 512 via flow controllers 516. The vacuum chamber 502 is also connected to vacuum pump 518, vacuum valve 522, and vent valve 520 which allow for pressure control of the vacuum chamber 502.

A radiofrequency (RF) generator 508 provides suitable power for igniting and sustaining plasmas 504 between the powered electrode 510 and grounding electrode 524. The power of the RF generator depends on the particular PECVD system used. In some embodiments, the power of the RF generator is between 0 W to 1000 W with frequency of 13.56 MHz. A person skilled in the art would understand that other RF generators operating at different frequencies and power levels may be used, as long as such generators can ignite and sustain the plasma for the applications described herein. The plasma 504 interacts with the precursor chemical 512 to deposition material on the surgical tool components 506. In an embodiment where the surgical tool components 506 are jaw components of an end effector, tissue treating surfaces (e.g., tissue treating surfaces 302, 304 shown in FIG. 3) are coated with the PDMS-like coating. In some embodiments, the PDMS-like coating covers a surface area on the jaw components 208, 210 or on the end effector 204 that is larger than the surface area of the tissue treating surfaces 302, 304.

An exemplary process 550 for PECVD is shown in FIG. 5B. In a first step 552, the components or tools to be coated are placed inside. The pressure in the vacuum chamber 502 is then reduced (554) to a predetermined pressure using the vacuum pump 518. In some embodiments, the predetermined pressure is approximately 50 mTorr to 300 mTorr. In a third step 556, after the pressure in the vacuum chamber 502 is reduced to the predetermined pressure, gas flows into the vacuum chamber 502 to reach a predetermined pressure, an oxygen plasma is used to clean and activate the surfaces (e.g., tissue treating surfaces 302, 304 of end effector 204 shown in FIG. 3) to prepare the surgical tool components 506 for coating depositions. This oxygen plasma treatment cleans and activates the sample substrate prior to deposition of the coating. In some embodiments, plasma of other gases or mixtures of gases may also be used for this purpose. In a fourth step 558, the chamber pressure is reduced again by pumping out the gases and reactive species. In a fifth step 560, the precursor chemical 512, which in the present embodiment is HMDSO, is delivered to the vacuum chamber 502 in a vapor phase. The RF generator 508 delivers RF energy to the electrode 510 to create plasma and begin the chemical deposition on the surface of the surgical tool components 506.

The deposition process is controlled by the generator power, time, flow rate, chamber pressure, and chamber temperature. In some embodiments, the chamber temperature is at approximately room temperature, for example, approximately between 18° C. to 31° C. In some embodiments, the chamber temperature is above room temperature, for example, approximately between 32° C. to 100° C. With power, flow rate, chamber pressure, and chamber temperature controlled, the coating thickness is determined based on the amount of time that the deposition process is run. For example, a coating deposition rate may be approximately 7 nm/min. For example, the overall time of the deposition process may be approximately 90 seconds to achieve a coating thickness of approximately 10 nm and may be approximately 40 minutes to achieve a coating thicknesses of approximately 250 nm. In some embodiments, during step 560, a carrier gas such as O₂ or Argon can be delivered together with HMDSO into the chamber. One of skill in the art would understand that it is possible to use other carrier gases during the deposition step 560 to adjust the deposition rate. It is understood that the deposition rate may be adjusted based on the process parameters described above.

In a sixth step 562, after the deposition process is completed and the desired coating thickness is achieved, the pressure of the vacuum chamber 502 is reduced to remove extra material from the chamber. Depending on the precursor material and the deposition processes of 560, a purge process may be added between the delivery and deposition step 562 and the venting step 564, wherein the gases and reactive species are pumped out to the exhaust before the venting step 564. The vacuum chamber 502 is then vented (562) with N₂ or air to atmospheric pressure, which allows the surgical tool components 506 to be removed from the vacuum chamber 502. In some embodiments, after the surgical tool components 506 are coated and removed from the vacuum chamber 502, the surgical tool components 506 are assembled to form an end effector of a surgical tool (e.g., end effector 204 of surgical tool 200 shown in FIGS. 2-3). In the surgical tool 200 shown in FIGS. 2-3, the jaw components 208, 210 can be assembled to form the jaws of the end effector 204 after the PDMS-like coating is deposited. In some embodiment, the end effector 204 can be coated directly in vacuum chamber 502 with the tissue treating surfaces 304 and/or 302 exposed to plasma.

FIGS. 6A and 6B show two apparatuses and methods for applying a second layer of the multi-layer anti-stick coating of FIG. 1. FIG. 6A shows a setup 600 for dipping a surgical tool 602 in a vessel 604 containing a phospholipid material 606 that forms a second layer of the multi-layer anti-stick coating when applied to the surgical tool 602. The surgical tool 602 is dipped into the vessel 604 containing the phospholipid material 606 to coat the end effector 608, and in some instances, at least a portion of a shaft 610 of the surgical tool 602. In some instances, the surgical tool 602 is allowed to air dry or oven dry. In other instances, excess phospholipid material may be wiped from the end effector 608 and/or shaft 610. In some instances, the thickness of the second layer of the multi-layer anti-stick coating is 300 nm to 5 μm.

While the above description of a dip coating process for a second layer of the multi-layer anti-stick coating is described with respect to a coating formed of a phospholipid material, a similar process may be used for a second layer formed of a silicone material. In some instances, the second layer formed of a silicone material has a thickness of approximately 300 nm to 5 μm, preferably 500 nm to 3 μm, over at least a portion of the sealing surface of the surgical tool. In some embodiments, at least 10, 20, 30, 40, 50, 60, 70, 80, 90, 95, 98, or 100 percent of the sealing surface of the surgical tool has a second layer formed of a silicone material with a thickness of approximately 300 nm to 5 μm, preferably 500 nm to 3 μm. In some embodiments, the second layer formed of a silicone material has a thickness of 0.1-1 μm (or, in some embodiments, 0.2-1 μm) over approximately 50-95% of the sealing surface area to which the second coating has been applied, and a thickness of greater than 7 μm over less than 5% of the sealing surface area to which the second coating has been applied (e.g., less than 5%, 4%, 3%, 2%, or 1%). In certain embodiments, the second coating has a thickness of greater than 1 μm to 2 μm over approximately 3-10% or approximately 25-35% of the sealing surface area to which the second coating has been applied. In particular embodiments, the second coating has a thickness distribution over the area to which the second coating has been applied that meets one of the embodiments in Table A.

While the above description of a dip coating process for a second layer of the multi-layer anti-stick coating is described with respect to coating a fully-assembled surgical tool, the dip coating process may also be performed on individual components and/or subassemblies (e.g., an assembled shaft) of a surgical tool.

FIG. 6B shows a setup 650 for wiping a second layer of the multi-layer anti-stick coating onto a surgical tool 652. In some embodiments, a cloth 658 is dipped into a vessel 654 containing a phospholipid material 656. The cloth 658 absorbs or otherwise carries the phospholipid material 656. The cloth 658 is used to wipe the phospholipid material 656 onto the end effector 660, and in some instances, at least a portion of a shaft 662 of the surgical tool 652. The wiping process produces a second layer of the multi-layer anti-stick coating that is 500 nm to 50 μm thick. In some instances, the thickness of the second layer of the multi-layer anti-stick coating is 500 nm to 5 μm. In some instances, the phospholipid material 656 is wiped onto tissue treating surfaces of the end effector 660 (e.g., tissue treating surfaces 302, 304 shown in FIG. 3). In some instances, the phospholipid material 656 is wiped over all surfaces coated by the PDMS-like material. In some instances, the phospholipid material 656 is wiped onto a smaller surface area of the surgical tool 652 than is covered by the PDMS-like material.

While the above description of a wiping coating process for a second layer of the multi-layer anti-stick coating is described with respect to a second layer formed of a phospholipid material, a similar process may be used for a coating formed of a silicone material.

While the above description of a wiping coating process for a second layer of the multi-layer anti-stick coating is described with respect to coating a fully-assembled surgical tool, the wiping coating process may also be performed on individual components and/or subassemblies (e.g., an assembled shaft) of a surgical tool.

In some embodiments, the phospholipid material that forms the second layer of the multi-layer anti-stick coating can be applied by spraying the phospholipid material onto the surgical tool (e.g., surgical tool 602 shown in FIG. 6A). The spraying process produces a second layer of the multi-layer anti-stick coating that is 500 nm to 30 μm thick. In some instances, the thickness of the second layer of the multi-layer anti-stick coating is between 300 nm to 5 μm. In some instances, the phospholipid material is sprayed over all surfaces coated by the PDMS-like material. In some instances, the phospholipid material is sprayed onto a smaller surface area of the surgical tool than is covered by the PDMS-like material.

While the above description of a spray coating process for a second layer of the multi-layer anti-stick coating is described with respect to a second layer formed of a phospholipid material, a similar process may be used for a coating formed of a silicone material.

While the above description of a spray coating process for a second layer of the multi-layer anti-stick coating is described with respect to coating a fully-assembled surgical tool, the spray coating process may also be performed on individual components and/or subassemblies (e.g., an assembled shaft) of a surgical tool.

In some embodiments, instead of an assembled surgical tool being dipped, wiped, or sprayed with the phospholipid material to form the second layer of the multi-layer anti-stick coating, the phospholipid material can be applied to components (e.g., jaw components 208, 210 of surgical tool 200 of FIGS. 2-3) of a surgical tool. In these embodiments, the components can be assembled after both of the first and second coatings have been applied.

As summarized below in Table 1, benchtop sticking tests were performed with vessel sealing surgical tools with a variety of coatings. Each vessel sealer (e.g., surgical tool 200 shown in FIG. 2) was activated to perform a sealing cycle which applies electrical energy to tissue. In some embodiments, the electrical energy can be delivered with the Ethicon GEN11 Generator. One of skill in the art would understand that other generators may be used for RF energy delivery. In some embodiments, the vessel sealer may deliver between approximately 20 to 210, such as 60 and 175, joules to tissue during a seal cycle. In some embodiments, the vessel sealer delivers between approximately 40 to 210 joules of energy to tissue during a seal cycle or between approximately 20 to 180 joules of energy to tissue during a seal cycle. Examples include approximately 20, 30, 40, 50, 60, 70, 80, 90, 100, 110, 120, 130, 140, 150, 160, 170, 180, 190, 200, and 210 joules of energy to tissue during a seal cycle.

After the sealing cycle is complete, the jaws of the vessel sealer were opened. If the tissue fell from the device jaws upon opening without manipulation by an operator of the test, the outcome was recorded as an anti-sticking event. If the tissue did not fall from the device jaws upon opening and required some device manipulation (jaws opening, jaw closing, and/or rotation) or if the tissue required the operator to remove it by hand or with another tool, the outcome was recorded as a sticking event. Each one of these tests that includes grasping tissue, running a sealing cycle, opening the jaws, and as necessary, manipulating the vessel sealer or manually removing tissue, is considered one activation.

Table 1, below, shows results of activations on porcine tissue for a variety of devices. Each device was an Ethicon RF Tissue Sealing device on which a large number of activations were performed, which is typical of clinical use. The first device was coated with a coating of an amino-functional silicone via a dip coating process. The second device was an Ethicon RF Tissue Sealer device that was coated with a 250 nm thick PDMS-like layer via PECVD as shown and described above with respect to FIGS. 5A-5B. The third device was coated with a phospholipid layer with a thickness of 1-3 μm as shown and described above with respect to FIGS. 6A-6B. It is important to note that this device did not include the PDMS-like first layer described above, only the phospholipid layer. The fourth device was coated with a first layer of a 250 nm thick PDMS-like material via PECVD and a second layer of 1-3 μm phospholipid material, as shown in FIG. 1 and described with respect to FIGS. 4-6B.

The amino-functional silicone coated device had an anti-sticking rate of 0.06. The device coated with only PECVD PDMS-like material had an anti-sticking rate of 0.2475 and the device coated with the phospholipid layer only had an anti-sticking rate of 0.2860. Each of these devices showed improved performance over the amino-functional silicone coated device. The combination of a first layer of PECVD PDMS-like material and a second layer of the phospholipid material on top produced an anti-sticking rate of 0.6000.

TABLE 1
Anti-Sticking Rate

| Device | Mean | Std Dev |
|---|---|---|
| Amino-Functional | 0.0600 | 0.0394 |
| Silicone | | |
| PECVD PDMS Only | 0.2475 | 0.0871 |
| Lipid Only | 0.2860 | 0.1161 |
| PECVD PDMS + Lipid | 0.6000 | 0.1395 |

It is important that the sealing capability of the vessel sealer be maintained even with the application of the anti-stick coating on the tissue treating surfaces of the vessel sealer. Table 2, below, shows results of burst testing on 5-7 mm porcine arteries. The porcine arteries had no tension placed on them and the test included 8 seals for 16 bursts per device. Each device was an Ethicon RF Tissue Sealer. The first device was coated with amino-functional silicone via dip coating. The second device was coated with a 250 nm thick PDMS-like layer via PECVD as shown and described above with respect to FIGS. 5A-5B. The third device was coated with a first layer of a 250 nm thick PDMS-like material via PECVD and a second layer of 300 nm to 50 μm phospholipid material, as shown in FIG. 1 and described with respect to FIGS. 4-6B.

The burst pressure results for each type of coated device was similar to the burst pressure performance of the amino-functional silicone coated device, meaning that the PDMS-like coating and the multi-layer anti-stick coating did not negatively affect sealing performance of the vessel sealer device.

TABLE 2
Burst Pressure (mmHg)

| Device | N | Mean | Std Dev |
|---|---|---|---|
| Amino-Functional | 80 | 704.4 | 321.3 |
| Silicone | | | |
| PECVD PDMS Only | 80 | 632.6 | 294.5 |
| PECVD PDMS + | 80 | 623.6 | 386.9 |
| Phospholipid | | | |

Table 3, below, shows results of activations on porcine tissue for a variety of devices. The first device group was that was coated with amino-functional silicone. The second device group was coated with an approximately 7 nm thick PDMS-like layer via PECVD as shown and described above with respect to FIGS. 5A-5B. A second layer consisting of a silicone material was then applied by dip coating on top of the 7 nm PECVD PDMS-like layer. The second layer had the following distribution of coating thickness along the sealing surfaces of the jaw: 0.1-1 μm (or, in some embodiments, 0.2-1 μm) over approximately 50-95% of the sealing surface area to which the second layer consisting of a silicone material has been applied, and a thickness of greater than 7 μm over less than 5% of the sealing surface area to which the second layer consisting of a silicone material has been applied (e.g., less than 5%, 4%, 3%, 2%, or 1%). In certain embodiments, the second coating has a thickness of greater than 1 μm to 2 μm over approximately 3-10% or approximately 25-35% of the sealing surface area to which the second layer consisting of a silicone material has been applied. In particular embodiments, the second layer consisting of a silicone material has a thickness distribution over the area to which the second layer consisting of a silicone material has been applied that meets one of the embodiments in Table A.

The device group coated with a layer of amino-functional silicone through dip coating had an anti-sticking rate of 3.5%, the device group coated with 7 nm thick PECVD PDMS plus a second layer of Silicone 2 had an anti-sticking rate of 43.7%, and the device group coated with 10 nm thick PECVD PDMS plus a second layer of Silicone 2 had an anti-sticking rate of 58%. Both PECVD PDMS plus a second layer of Silicone 2 show significant improvement in anti-sticking performance over the control amino-functional silicone only group. The method used for this testing categorizes any event where tissue that takes longer than 2 seconds to fall from the end effector as a sticking event.

TABLE 3
Anti-Sticking Rate

| Device | Mean | Std Dev |
|---|---|---|
| Amino-functional | 0.0350 | 0.0193 |
| silicone coated | | |
| PECVD PDMS | 0.4367 | 0.0808 |
| 7 nm + Silicone 2 | | |
| PECVD PDMS | 0.5800 | 0.01414 |
| 10 nm + Silicone 2 | | |

One skilled in the art will appreciate further features and advantages of the devices, systems, and methods based on the above-described embodiments. Accordingly, this disclosure is not to be limited by what has been particularly shown and described, except as indicated by the appended claims. All publications and references cited herein are expressly incorporated herein by reference in their entirety for all purposes.

The present disclosure has been described above by way of example only within the context of the overall disclosure provided herein. It will be appreciated that modifications within the spirit and scope of the claims may be made without departing from the overall scope of the present disclosure.

What is claimed is:

1. A method of manufacturing a surgical tool, the method comprising:
forming, using plasma enhanced chemical vapor deposition with a precursor material, a first coating on a conductive tissue treating surface of a component of an end effector of the surgical tool, the first coating comprising a first material, wherein the first material is a first silicone material; and
applying a second coating on top of the first coating on the conductive tissue treating surface, the second coating comprising a second material,
wherein the first coating and the second coating form a multi-layer coating that is effective to prevent tissue sticking to the conductive tissue treating surface during an electrosurgical sealing procedure.

2. The method of claim 1, wherein the precursor used for plasma enhanced chemical vapor deposition is hexamethyldisiloxane.

3. The method of claim 1, wherein the first silicone material is a polydimethylsiloxane-like material.

4. The method of claim 1, wherein the first silicone material comprises polydimethylsiloxane.

5. The method of claim 1, wherein the second material comprises a phospholipid material.

6. The method of claim 1, wherein the second material comprises a second silicone material different from the first silicone material.

7. The method of claim 6, wherein the second silicone material comprises an amino-functional silicone.

8. The method of claim 1, wherein applying the second coating comprises wiping the second material onto the conductive tissue treating surface.

9. The method of claim 1, wherein applying the second coating comprises spraying the second material onto the conductive tissue treating surface.

10. The method of claim 1, wherein applying the second coating comprises brushing the second material onto the conductive tissue treating surface.

11. The method of claim 1, wherein applying the second coating comprises dipping the conductive tissue treating surface into the second material.

12. The method of claim 1, wherein the component is a first jaw component of the end effector.

13. The method of claim 12, further comprising, after forming the first coating and prior to applying the second coating, assembling the end effector using the first jaw component and a second jaw component.

14. The method of claim 12, further comprising, after applying the second coating, assembling the end effector using the first jaw component and a second jaw component.

15. The method of claim 1, wherein the first coating has a thickness of approximately 7 to 17 nm or 220 to 300 nm.

16. The method of claim 15, wherein the first coating has a thickness of approximately 7 to 17 nm.

17. The method of claim 15, wherein the first coating has a thickness of approximately 220 to 300 nm.

18. The method of claim 1, wherein the second coating has a thickness of 300 nm to 5 μm.

19. The method of claim 1, wherein the second coating has a thickness of 0.1-1 μm over approximately 50-95% of the sealing surface area to which the second coating has been applied, and a thickness of greater than 7 μm over less than 5% of the sealing surface area to which the second coating has been applied.

20. An end effector of an electrosurgical device, the end effector comprising:
a first jaw component having a conductive first tissue treating surface;
a second jaw component operatively coupled to the first jaw component, the second jaw component having a conductive second tissue treating surface;
a first coating on the conductive first tissue treating surface and the conductive second tissue treating surface, the first coating comprising a first material that is a first silicone material; and
a second coating layered on top of the first coating, the second coating comprising a second material.
